SEQUENCE LISTING (25 sequences)

SEQ ID NO: 1 - protein, 118 aa, Artificial Sequence
  Peptide inhibitor of caspase-9 is XBIR3
MGSSHHHHHHSSGLVPRGSHMSTNTCLPRNPSMADYEARIFTFGTWIYSVNKEQLARAGFYTDWALGEGDKVKCFHCGGGLRPSEDPWEQHARWYPGCRYLLEQRGQEYINNIHLTHS

SEQ ID NO: 2 - protein, 112 aa, Artificial Sequence
  Peptide inhibitor of caspase-9 is XBIR3
MGSSSSGLVPRGSHMSTNTCLPRNPSMADYEARIFTFGTWIYSVNKEQLARAGFYTDWALGEGDKVKCFHCGGGLRPSEDPWEQHARWYPGCRYLLEQRGQEYINNIHLTHS

SEQ ID NO: 3 - protein, 108 aa, Artificial Sequence
  Peptide inhibitor of caspase-9 is XBIR3
SSGLVPRGSHMSTNTCLPRNPSMADYEARIFTFGTWIYSVNKEQLARAGFYTDWALGEGDKVKCFHCGGGLRPSEDPWEQHARWYPGCRYLLEQRGQEYINNIHLTHS

SEQ ID NO: 4 - protein, 98 aa, Artificial Sequence
  Peptide inhibitor of caspase-9 is XBIR3
MSTNTCLPRNPSMADYEARIFTFGTWIYSVNKEQLARAGFYTDWALGEGDKVKCFHCGGGLRPSEDPWEQHARWYPGCRYLLEQRGQEYINNIHLTHS

SEQ ID NO: 5 - protein, 117 aa, Artificial Sequence
  Peptide inhibitor of caspase-9 is XBIR3
MGSSHHHHHHSSGLVPRGSHMSTNTLPRNPSMADYEARIFTFGTWIYSVNKEQLARAGFYTDWALGEGDKVKCFHCGGGLRPSEDPWEQHARWYPGCRYLLEQRGQEYINNIHLTHS

SEQ ID NO: 6 - protein, 111 aa, Artificial Sequence
  Peptide inhibitor of caspase-9 is XBIR3
MGSSSSGLVPRGSHMSTNTLPRNPSMADYEARIFTFGTWIYSVNKEQLARAGFYTDWALGEGDKVKCFHCGGGLRPSEDPWEQHARWYPGCRYLLEQRGQEYINNIHLTHS

SEQ ID NO: 7 - protein, 107 aa, Artificial Sequence
  Peptide inhibitor of caspase-9 is XBIR3
SSGLVPRGSHMSTNTLPRNPSMADYEARIFTFGTWIYSVNKEQLARAGFYTDWALGEGDKVKCFHCGGGLRPSEDPWEQHARWYPGCRYLLEQRGQEYINNIHLTHS

SEQ ID NO: 8 - protein, 97 aa, Artificial Sequence
  Peptide inhibitor of caspase-9 is XBIR3
MSTNTLPRNPSMADYEARIFTFGTWIYSVNKEQLARAGFYTDWALGEGDKVKCFHCGGGLRPSEDPWEQHARWYPGCRYLLEQRGQEYINNIHLTHS

SEQ ID NO: 9 - protein, 17 aa, Artificial Sequence
  cell-penetrating peptide of the membrane-permeable complex is Penetratin1
  mod_res (1)..(2): NPys
CRQIKIWFQNRRMKWKK

SEQ ID NO: 10 - protein, 16 aa, Artificial Sequence
  Exemplary cell permeant molecule RL16 a sequence derived from Penetratin1
RRLRRLLRRLLRRLRR

SEQ ID NO: 11 - protein, 12 aa, Artificial Sequence
  Exemplary cell permeant molecule - rabies virus sequence which targets neurons
RVGRRRRRRRRR

SEQ ID NO: 12 - protein, 27 aa, Artificial Sequence
  Synthetic sequence
GWTLNSAGYLLGKINLKALAALAKKIL

SEQ ID NO: 13 - protein, 16 aa, Artificial Sequence
  Synthetic sequence - amino acid sequence included in pIsl
PVIRVWFQNKRCKDKK

SEQ ID NO: 14 - protein, 13 aa, Artificial Sequence
  Synthetic sequence - small Tat fragment
GRKKRRQRRRPPQ

SEQ ID NO: 15 - protein, 16 aa, Artificial Sequence
  Synthetic sequence - amino acid sequence included in pVEC
LLIILRRRIRKQAHAH

SEQ ID NO: 16 - protein, 27 aa, Artificial Sequence
  Synthetic sequence - amino acid sequence included in MPS
GALFLGWLGAAGSTMGAWSQPKKKRKV

SEQ ID NO: 17 - protein, 18 aa, Artificial Sequence
  Synthetic sequence - exemplary MAP sequence
KLALKLALKALKAALKLA

SEQ ID NO: 18 - protein, 135 aa, Artificial Sequence
  Synthetic sequence caspase-9 signaling pathway inhibitor comprising XBIR3 conjugated to a cell-penetrating peptide
  mod_res (1)..(2): NPys
  mod_res (17)..(18): disulfide bond
CRQIKIWFQNRRMKWKKMGSSHHHHHHSSGLVPRGSHMSTNTCLPRNPSMADYEARIFTFGTWIYSVNKEQLARAGFYTDWALGEGDKVKCFHCGGGLRPSEDPWEQHARWYPGCRYLLEQRGQEYINNIHLTHS

SEQ ID NO: 19 - protein, 129 aa, Artificial Sequence
  Synthetic sequence caspase-9 signaling pathway inhibitor comprising XBIR3 conjugated to a cell-penetrating peptide
  mod_res (1)..(2): NPys
  mod_res (17)..(18): disulfide bond
CRQIKIWFQNRRMKWKKMGSSSSGLVPRGSHMSTNTCLPRNPSMADYEARIFTFGTWIYSVNKEQLARAGFYTDWALGEGDKVKCFHCGGGLRPSEDPWEQHARWYPGCRYLLEQRGQEYINNIHLTHS

SEQ ID NO: 20 - protein, 125 aa, Artificial Sequence
  Synthetic sequence caspase-9 signaling pathway inhibitor comprising XBIR3 conjugated to a cell-penetrating peptide
  mod_res (1)..(2): NPys
  mod_res (17)..(18): disulfide bond
CRQIKIWFQNRRMKWKKSSGLVPRGSHMSTNTCLPRNPSMADYEARIFTFGTWIYSVNKEQLARAGFYTDWALGEGDKVKCFHCGGGLRPSEDPWEQHARWYPGCRYLLEQRGQEYINNIHLTHS

SEQ ID NO: 21 - protein, 114 aa, Artificial Sequence
  Synthetic sequence caspase-9 signaling pathway inhibitor comprising XBIR3 conjugated to a cell-penetrating peptide
  mod_res (1)..(1): NPys
  mod_res (16)..(17): disulfide bond
RQIKIWFQNRRMKWKKMSTNTCLPRNPSMADYEARIFTFGTWIYSVNKEQLARAGFYTDWALGEGDKVKCFHCGGGLRPSEDPWEQHARWYPGCRYLLEQRGQEYINNIHLTHS

SEQ ID NO: 22 - protein, 134 aa, Artificial Sequence
  Synthetic sequence caspase-9 signaling pathway inhibitor comprising XBIR3 conjugated to a cell-penetrating peptide
  mod_res (1)..(2): NPys
  mod_res (17)..(18): disulfide bond
CRQIKIWFQNRRMKWKKMGSSHHHHHHSSGLVPRGSHMSTNTLPRNPSMADYEARIFTFGTWIYSVNKEQLARAGFYTDWALGEGDKVKCFHCGGGLRPSEDPWEQHARWYPGCRYLLEQRGQEYINNIHLTHS

SEQ ID NO: 23 - protein, 128 aa, Artificial Sequence
  Synthetic sequence caspase-9 signaling pathway inhibitor comprising XBIR3 conjugated to a cell-penetrating peptide
  mod_res (1)..(2): NPys
  mod_res (17)..(18): disulfide bond
CRQIKIWFQNRRMKWKKMGSSSSGLVPRGSHMSTNTLPRNPSMADYEARIFTFGTWIYSVNKEQLARAGFYTDWALGEGDKVKCFHCGGGLRPSEDPWEQHARWYPGCRYLLEQRGQEYINNIHLTHS

SEQ ID NO: 24 - protein, 124 aa, Artificial Sequence
  Synthetic sequence caspase-9 signaling pathway inhibitor comprising XBIR3 conjugated to a cell-penetrating peptide
  mod_res (1)..(2): NPys
  mod_res (17)..(18): disulfide bond
CRQIKIWFQNRRMKWKKSSGLVPRGSHMSTNTLPRNPSMADYEARIFTFGTWIYSVNKEQLARAGFYTDWALGEGDKVKCFHCGGGLRPSEDPWEQHARWYPGCRYLLEQRGQEYINNIHLTHS

SEQ ID NO: 25 - protein, 114 aa, Artificial Sequence
  Synthetic sequence caspase-9 signaling pathway inhibitor comprising XBIR3 conjugated to a cell-penetrating peptide
  mod_res (1)..(2): NPys
  mod_res (17)..(18): disulfide bond
CRQIKIWFQNRRMKWKKMSTNTLPRNPSMADYEARIFTFGTWIYSVNKEQLARAGFYTDWALGEGDKVKCFHCGGGLRPSEDPWEQHARWYPGCRYLLEQRGQEYINNIHLTHS

(19) United States
(12) Patent Application Publication
(10) Pub. No.: US 2024/0197895 A1
(43) Pub. Date: Jun. 20, 2024

(54) COMPOSITIONS AND METHODS FOR TREATING PATIENTS WITH MITOCHONDRIAL COMPLEX I DEFICIENCY USING CASPASE-9 SIGNALING PATHWAY INHIBITORS

(71) Applicant: THE TRUSTEES OF COLUMBIA UNIVERSITY IN THE CITY OF NEW YORK, New York, NY (US)

(72) Inventors: Carol M. TROY, Hasting-on-Hudson, NY (US); Maria I. AVRUTSKY, Burlingame, CA (US)

(21) Appl. No.: 18/258,796

(22) PCT Filed: Dec. 14, 2021
(86) PCT No.: PCT/US2021/063363

§ 371 Date: Jun. 21, 2023

Related U.S. Application Data
(60) Provisional application No. 63/129,197, filed on Dec. 22, 2020.

(51) Int. Cl. A61K 47/64; A61K 9/00; A61K 38/17; A61K 47/69; A61P 27/02

(52) U.S. Cl. CPC A61K 47/645; A61K 9/0048; A61K 38/1761; A61K 47/6925; A61P 27/02

(57) ABSTRACT

The present disclosure relates to a method for treating mitochondrial complex I deficiency comprising administering to patients with mitochondrial complex I deficiency an effective amount of XBIR3. The effective amount of XBIR 3 may be conjugated to a cell-penetrating peptide, such conjugation may include encapsulation of XBIR3 in nano-carrier that is conjugated to a cell penetrating peptide or direct conjugation of XBIR3 to a cell penetrating peptide. The XBIR-3 conjugated to a cell-penetrating peptide may be administered directly to the eye of the patient, administered systemically, or administered intranasally.

This application claims the benefit of priority to U.S. Provisional Application No. 63/129,197, filed Dec. 22, 2020, which application is hereby incorporated by reference in its entirety.

### TECHNICAL FIELD

The present disclosure relates to methods for the treatment and inhibition of mitochondrial complex I deficiency.

### STATEMENT REGARDING SEQUENCE LISTING

The Sequence Listing associated with this application is provided in text format in lieu of a paper copy, and is hereby incorporated by reference into the specification. The name of the text file containing the Sequence Listing is 210271_41101WO_SEQUENCE_LISTING.txt. The text file is 22.3 KB, was created on Dec. 13, 2021, and is being submitted electronically via EFS-Web.

### BACKGROUND

Mitochondrial diseases are rare inherited disorders caused by deficits in mitochondrial function. Central nervous system (CNS) tissues are especially affected in mitochondrial disease due to high energy demands; consequently, CNS degeneration, subsequent neurological deficits and vision loss are a common feature of these disorders.

Mutations causing mitochondrial complex I deficiency underlie several mitochondrial disorders, including Leber's Hereditary Optic Neuropathy, Leigh Syndrome, and autosomal dominant optic atrophy. The NDUFS4 enzyme is an essential component of mitochondrial complex I, and NDUFS4 knock-out (KO) mice have severe deficits in complex I function, causing fatal early onset neurodegeneration representative of Leigh Syndrome.

A mitochondrial complex I deficiency is caused by a shortage or a loss of function of a protein complex called complex I. Mitochondrial complex I deficiency can cause many neuroglial issues in a patient, such as seizures, abnormal brain function, and involuntary movements. Mitochondrial complex I deficiency also can cause vision problems. These vision problems are due to breakdown of the optic nerves that carry signals from the eyes to the brains.

### SUMMARY

The present disclosure provides methods for treating a patient with mitochondrial complex I deficiency by administering XBIR3 conjugated to a cell-penetrating peptide, in an amount effective to treat mitochondrial complex I deficiency. An effective amount of XBIR3 may be in a concentration between 0.1 μM and 1,000 μM, inclusive. The methods may further include administering to the eye of a patient the XBIR3 conjugated to a cell-penetrating peptide. The administration to the eye may include administration by using an eye drop or a topical ophthalmic ointment. Further methods of administration may include systemic or intranasal delivery of XBIR3 conjugated to a cell-penetrating peptide to the patient.

The cell-penetrating peptide, may be selected from a group consisting of Penetratin1, transportan, pISI, Tat(48-60), pVEC, MAP, and MTS. And, XBIR3 may be conjugated to the cell-penetrating peptide via a disulfide bond. XBIR3 may also be indirectly conjugated to the cell-penetrating peptide, by encapsulating the XBIR within a nano-carrier, and the nano-carrier is conjugated to a cell-penetrating peptide.

The methods disclosed may treat Mitochondrial Complex I by a variety of non-exclusive means including: (1) decreasing the amount of 4-HNE in the inner plexiform layer of the eye, (2) increasing the thickness of the inner plexiform layer of the eye, (3) decreasing the amount of cl-Casp-9 in the inner plexiform layer of the eye, (4) increasing the patient's contrast sensitivity, and (5) increasing the patient's acuity sensitivity.

The present disclosure further provides a method for treating a mitochondrial complex I deficiency, the method comprising administering to a patient having a mitochondrial complex I deficiency an amount of a caspase-9 signaling pathway inhibitor comprising XBIR3 conjugated to a cell-penetrating peptide effective to treat the mitochondrial complex I deficiency.

In more specific embodiments, which may be combined with one another unless clearly mutually exclusive:
administering comprises administering to an eye, containing an inner plexiform layer, of the patient;
administering to the eye of the patient comprises administering an eye drop comprising the caspase-9 signaling pathway inhibitor comprising XBIR3 conjugated to a cell-penetrating peptide;
administering to the eye of the patient comprises administering a topical ophthalmic ointment comprising the caspase-9 signaling pathway inhibitor comprising XBIR3 conjugated to a cell-penetrating peptide;
administering comprises administering systemically to the patient;
administering comprises administering via intranasal delivery to the patient;
the cell-penetrating peptide is selected from the group consisting of Penetratin1, transportan, pIS1, Tat(48-60), pVEC, MAP, and MTS;
the XBIR3 is conjugated to a cell-penetrating peptide via a disulfide bond;
the amount of a caspase-9 signaling pathway inhibitor comprising XBIR3 conjugated to a cell-penetrating peptide effective to treat the mitochondrial complex I deficiency decreases the amount of 4-Hydroxynonenal (4-HNE) in the inner plexiform layer of the eye of the patient by more than 5%;
the amount of a caspase-9 signaling pathway inhibitor comprising XBIR3 conjugated to a cell-penetrating peptide effective to treat the mitochondrial complex I deficiency reduces retinal thinning by at least 5%;
the amount of a caspase-9 signaling pathway inhibitor comprising XBIR3 conjugated to a cell-penetrating peptide effective to treat the mitochondrial complex I deficiency decreases the amount of cl-Casp-9 in the inner plexiform layer of the eye of the patient by more than 5%;
the amount of a caspase-9 signaling pathway inhibitor comprising XBIR3 conjugated to a cell-penetrating peptide effective to treat the mitochondrial complex I deficiency increases the patient's acuity sensitivity by more than 10 cycle/degrees as measured by visual acuity testing;
the amount of a caspase-9 signaling pathway inhibitor comprising XBIR3 conjugated to a cell-penetrating peptide effective to treat the mitochondrial complex I deficiency increases the patient's contrast sensitivity as measured by an improvement of 10% minimum contrast detected as measured by contrast sensitivity testing;
the caspase-9 signaling pathway inhibitor comprising XBIR3 conjugated to a cell-penetrating peptide is administered at a concentration between 0.1 μM and 1,000 μM, inclusive;
XBIR3 is indirectly conjugated to a cell-penetrating peptide, by encapsulating XBIR3 within a nano-carrier, and the nano-carrier is conjugated to a cell-penetrating peptide;
the amount of a caspase-9 signaling pathway inhibitor comprising XBIR3 conjugated to a cell-penetrating peptide effective to treat the mitochondrial complex I deficiency decreases the rate of ganglion cell death as measured by an improvement of 5% minimum contrast detected as measured by contrast sensitivity testing;
the XBIR3 has a sequence selected from the group consisting of SEQ ID NOs: 1-8;
the cell-penetrating peptide has a sequence selected from the group consisting of SEQ ID NOs: 9-17;
the caspase-9 signaling pathway inhibitor comprising XBIR3 conjugated to a cell-penetrating peptide has a sequence selected from:

C(NPys)-RQIKIWFQNRRMKWKK-s-s-
(SEQ ID NO: 18)
MGSSHHHHHHSSGLVPRGSHMSTNTCLPRNPSMADYEARIFTFGTWIYSV
NKEQLARAGFYTDWALGEGDKVKCFHCGGGLRPSEDPWEQHARWYPGCRY
LLEQRGQEYINNIHLTHS;
C(NPys)-RQIKIWFQNRRMKWKK-s-s-
(SEQ ID NO: 19)
MGSSSSGLVPRGSHMSTNTCLPRNPSMADYEARIFTFGTWIYSVNKEQLA
RAGFYTDWALGEGDKVKCFHCGGGLRPSEDPWEQHARWYPGCRYLLEQRG
QEYINNIHLTHS;
C(NPys)-RQIKIWFQNRRMKWKK-s-s-
(SEQ ID NO: 20)
SSGLVPRGSHMSTNTCLPRNPSMADYEARIFTFGTWIYSVNKEQLARAGF
YTDWALGEGDKVKCFHCGGGLRPSEDPWEQHARWYPGCRYLLEQRGQEYI
NNIHLTHS;
(NPys)-RQIKIWFQNRRMKWKK-s-s-
(SEQ ID NO: 21)
MSTNTCLPRNPSMADYEARIFTFGTWIYSVNKEQLARAGFYTDWALGEGD
KVKCFHCGGGLRPSEDPWEQHARWYPGCRYLLEQRGQEYINNIHLTHS;
C(NPys)-RQIKIWFQNRRMKWKK-s-s-
(SEQ ID NO: 22)
MGSSHHHHHHSSGLVPRGSHMSTNTLPRNPSMADYEARIFTFGTWIYSVN
KEQLARAGFYTDWALGEGDKVKCFHCGGGLRPSEDPWEQHARWYPGCRYL
LEQRGQEYINNIHLTHS;
C(NPys)-RQIKIWFQNRRMKWKK-s-s-
(SEQ ID NO: 23)
MGSSSSGLVPRGSHMSTNTLPRNPSMADYEARIFTFGTWIYSVNKEQLAR
AGFYTDWALGEGDKVKCFHCGGGLRPSEDPWEQHARWYPGCRYLLEQRGQ
EYINNIHLTHS;
C(NPys)-RQIKIWFQNRRMKWKK-s-s-
(SEQ ID NO: 24)
SSGLVPRGSHMSTNTLPRNPSMADYEARIFTFGTWIYSVNKEQLARAGFY
TDWALGEGDKVKCFHCGGGLRPSEDPWEQHARWYPGCRYLLEQRGQEYIN
NIHLTHS;
and
C(NPys)-RQIKIWFQNRRMKWKK-s-s-
(SEQ ID NO: 25)
MSTNTLPRNPSMADYEARIFTFGTWIYSVNKEQLARAGFYTDWALGEGDK
VKCFHCGGGLRPSEDPWEQHARWYPGCRYLLEQRGQEYINNIHLTHS.

The present specification references various embodiments of the disclosure and provides various examples. These embodiments and examples may also be used in combination with one another and with any of the above methods unless they are clearly excluded therefrom.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure may be further understood through reference to the attached figures in combination with the detailed description that follows.

FIG. 1 is a graph showing weights for the wild type and NDUFS4 KO males and female groups of mice over time.

FIG. 2 is a series of graphs showing electroretinogram readouts for wild type and NDUFS4 KO mice over time. The electroretinogram measured A wave amplitude, B wave amplitude, and oscillatory potential amplitude.

FIG. 3 is a set of optical coherence tomography images of eyes of wild type and NDUFS4 KO mice, showing the effects of mitochondrial complex I deficiency on the inner plexiform layer of the eye in mice.

FIG. 4 is a graph showing the change in inner plexiform layer thickness over time in wild type and NDUFS4 KO mice.

FIG. 5 is graph of the change in visual acuity sensitivity of wild type and NDUFS4 KO mice over time and a visual representation of the acuity measure in the visual acuity sensitivity test.

FIG. 6 is graph of the change in visual contrast sensitivity of wild type and NDUFS4 KO mice over time and a visual representation of the contrast measure in the visual contrast sensitivity test.

FIG. 7 is a graph of the visual acuity sensitivity versus inner plexiform layer thickness for wild type and NDUFS4 KO mice.

FIG. 8 is a graph of the visual contrast sensitivity versus inner plexiform layer thickness for wild type and NDUFS4 KO mice.

FIG. 9 are representative images of TUNEL staining of the eyes of wild type and NDUFS4 KO mice. The TUNEL staining was preformed to look for the presence of cl-Casp-9 in the retina's different layers in the wild type and NUFS4 KO mice. The represented retina layers are ganglion cell layer (GCL), inner nuclear layer (INL), and outer nuclear layer (ONL).

FIG. 10 is a graph showing the correlation of Inner plexiform Layer (“IPL”) thickness versus the presence of cl-Casp-9, in wild type and NDUFS4 KO mice.

FIG. 11 is a graph showing the correlation of the contrast sensitivity data (represented as minimum contrast detected) versus the presence of cl-Casp-9, in wild type and NDUFS4 KO mice.

FIG. 12 is a series of graphs showing IPL and intraretinal thickness change in response to treatment with Pen1-XBir3 and Pen1-mutXBir3. Graph A shows inner plexiform layer thickness in NDUFS4 KO eyes treated with Pen1-mutXBir3 or Pen1-XBir3 measured by OCT imaging at prenatal day 35, 42, and 49. Dataset from an observational cohort of untreated NDUFS4 KO and NDUFS4 Wild Type eyes overlaid on graph for reference. Graph B shows percent change from prenatal day 42 to 49 in inner plexiform layer thickness in NDUFS4 KO eye treated with Pen1-mutXBir3 or Pen1-XBir3 measured by OCT imaging. Lastly, Graph C shows percent change from prenatal day 42 to 49 in intraretinal thickness in NDUFS4 KO eye treated with Pen1-mutXBir3 or Pen1-XBir3 measured by OCT imaging.

FIG. 13 is a series of graphs showing Acuity and Contrast data measured by optomotor reflex (“OMR”) in the OptoDrum (Striatech) automated optomotor system. Graph A shows the measured acuity in NDUFS4 KO mice treated with either Pen1-mutXBir3 or Pen1-XBir3 assessed before onset of treatment at prenatal day 35, and again at prenatal day 42 and 49. Graph B shows measured contrast in NDUFS4 KO mice treated with either Pen1-mutXBir3 or Pen1-XBir3 assessed before onset of treatment at prenatal day 35, and again at prenatal day 42 and 49. A dataset from an observational cohort of untreated NDUFS4 KO overlaid on graph A and B for reference. Lastly, Graph C shows the acuity change vs contrast change.

### DETAILED DESCRIPTION

The present disclosure relates to a method for treating mitochondrial complex I deficiency in a patient. For example, but not by way of limitation, the present disclosure relates to a method for inhibiting caspase-9 signaling activity associated with the induction and/or exacerbation of mitochondrial complex I deficiency in a patient.

As used herein, the term “mitochondrial complex I deficiency” refers to clinically detectable mitochondrial complex I deficiency. Clinical symptoms of mitochondrial complex I deficiency may include vision loss or impairment. Clinical symptoms of mitochondrial complex I deficiency may also include any of the following: abnormally low levels of mitochondrial complex I, a reduction of the inner plexiform layer of the eye which can measured by optical coherence tomography, visual acuity and contrast loss, and abnormally high levels of 4-HNE.

As used herein, the term “patient” refers to any animal, including any mammal, including, but not limited to, humans, and non-human animals (including, but not limited to, non-human primates, dogs, cats, rodents, horses, cows, pigs, mice, rats, hamsters, rabbits, and the like. In particular, the patient is a human.

As used herein, an “effective amount” or an “amount effective” is an amount sufficient to cause a beneficial or desired clinical result in a patient. An effective amount can be administered to a patient in one or more doses. It is typically administered to the retina of the patient. In terms of treatment, an effective amount is an amount that is sufficient to ameliorate the impact of and/or inhibit the induction and/or exacerbation of mitochondrial complex I deficiency in a patient, or otherwise reduce the pathological consequences of the disease(s). The effective amount is generally determined by the physician on a case-by-case basis and is within the skill of one in the art. Several factors may be taken into account when determining an appropriate dosage to achieve an effective amount. These factors include age, the condition being treated, the severity of the condition, prior responses, type of inhibitor used, the caspase-9 signaling pathway member to be inhibited, the cell type expressing the target, and the form and effective concentration of the composition (also referred to herein as a “treatment,” “inhibitor,” or “conjugate”) being administered.

As used herein, “treat,” “treating” and similar verbs refer to of ameliorating the impact of and/or inhibiting the induction and/or exacerbation of mitochondrial complex I deficiency in a patient.

### Methods of Inhibiting Mitochondrial Complex I Deficiency

In certain embodiments, the instant disclosure is directed to methods of or uses of treatments disclosed herein in ameliorating the impact of and/or inhibiting the induction and/or exacerbation of mitochondrial complex I deficiency in a patient by administering an effective amount of a caspase-9 signaling pathway inhibitor or conjugate thereof. In certain embodiments, the methods of the present disclosure are directed to the administration of a caspase-9 signaling pathway inhibitor, or conjugate thereof, via eye drops, eye ointments, or other ocular formulations, intra-ocular or systemic injection, or intranasal formulations in order to inhibit mitochondrial complex I deficiency.

The treatment, when used to treat the effects of mitochondrial complex I deficiency, may be administered as a single dose or multiple doses. For example, but not by way of limitation, where multiple doses are administered, they may be administered at intervals of 6 times per 24 hours or 4 times per 24 hours or 3 times per 24 hours or 2 times per 24 hours or 1 time per 24 hours or 1 time every other day or 1 time every 3 days or 1 time every 4 days or 1 time per week, or 2 times per week, or 3 times per week. In certain embodiments, the initial dose may be greater than subsequent doses or all doses may be the same.

In certain embodiments, the inhibitor used in connection with the methods and uses of the instant disclosure is a Pen1-XBIR3 conjugate as disclosed herein. In certain embodiments, the Pen1-XBIR3 conjugate is administered to a patient suffering from mitochondrial complex I deficiency either as a single dose or in multiple doses. The concentration of the Pen1-XBIR3 composition administered is, in certain embodiments: 0.1 μM to 1,000 μM; 1 μM to 500 μM; 10 μM to 100 μM; or 20 μM to 60 μM, inclusive. In certain embodiments, a specific human equivalent dosage can be calculated from animal studies via body surface area comparisons. In certain embodiments, eye size comparisons can be employed to calculate a specific human equivalent dosage.

In certain embodiments, the caspase-9 signaling pathway inhibitor, either alone or in the context of a membrane-permeable conjugate, is administered in conjunction with one or more additional therapeutics. In certain of such embodiments, the additional therapeutics include, but are not limited to an anti-VEGF therapeutic and/or a steroidal therapeutic. In certain embodiments, the method involves the administration of one or more additional caspase-9 signaling pathway inhibitors either alone or in the context of a membrane-permeable conjugate.

In certain embodiments, the caspase-9 signaling pathway inhibitor may treat mitochondrial complex I deficiency by decreasing the amount of 4-HNE or cl-Casp-9 in the inner plexiform layer of the eye of a patient. In certain embodiments, the caspase-9 signaling pathway inhibitor may treat mitochondrial complex I deficiency by decreasing apoptosis in the inner plexiform layer of the patient, as detected by optical coherence tomography.

### Caspase-9 Signaling Pathway Inhibitors

In certain embodiments, the caspase-9 signaling pathway inhibitors of the present disclosure are peptide inhibitors of caspase-9.

In certain embodiments, the peptide inhibitor of caspase-9 is XBIR3 having the sequence:

(SEQ ID NO. 1)
MGSSHHHHHHSSGLVPRGSHMSTNTCLPRNPSMADYEARIFTFGTWIYSV
NKEQLARAGFYTDWALGEGDKVKCFHCGGGLRPSEDPWEQHARWYPGCRY
LLEQRGQEYINNIHLTHS.

In certain embodiments, the peptide inhibitor of caspase-9 is XBIR3 having the sequence:

(SEQ ID NO. 2)
MGSSSSGLVPRGSHMSTNTCLPRNPSMADYEARIFTFGTWIYSVNKEQLA
RAGFYTDWALGEGDKVKCFHCGGGLRPSEDPWEQHARWYPGCRYLLEQRG
QEYINNIHLTHS.

In certain embodiments, the peptide inhibitor of caspase-9 is XBIR3 having the sequence:

(SEQ ID NO. 3)
SSGLVPRGSHMSTNTCLPRNPSMADYEARIFTFGTWIYSVNKEQLARAGF
YTDWALGEGDKVKCFHCGGGLRPSEDPWEQHARWYPGCRYLLEQRGQEYI
NNIHLTHS.

In certain embodiments, the peptide inhibitor of caspase-9 is XBIR3 having the sequence:

(SEQ ID NO. 4)
MSTNTCLPRNPSMADYEARIFTFGTWIYSVNKEQLARAGFYTDWALGEGD
KVKCFHCGGGLRPSEDPWEQHARWYPGCRYLLEQRGQEYINNIHLTHS.

In certain embodiments the peptide inhibitor of caspase-9 is XBIR3 having the

sequence:
(SEQ ID NO. 5)
MGSSHHHHHHSSGLVPRGSHMSTNTLPRNPSMADYEARIFTFGTWIYSVN
KEQLARAGFYTDWALGEGDKVKCFHCGGGLRPSEDPWEQHARWYPGCRYL
LEQRGQEYINNIHLTHS.

In certain embodiments the peptide inhibitor of caspase-9 is XBIR3 having the sequence:

(SEQ ID NO. 6)
MGSSSSGLVPRGSHMSTNTLPRNPSMADYEARIFTFGTWIYSVNKEQLAR
AGFYTDWALGEGDKVKCFHCGGGLRPSEDPWEQHARWYPGCRYLLEQRGQ
EYINNIHLTHS.

In certain embodiments the peptide inhibitor of caspase-9 is XBIR3 having the sequence:

(SEQ ID NO. 7)
SSGLVPRGSHMSTNTLPRNPSMADYEARIFTFGTWIYSVNKEQLARAGFY
TDWALGEGDKVKCFHCGGGLRPSEDPWEQHARWYPGCRYLLEQRGQEYIN
NIHLTHS.

In certain embodiments the peptide inhibitor of caspase-9 is XBIR3 having the sequence:

(SEQ ID NO. 8)
MSTNTLPRNPSMADYEARIFTFGTWIYSVNKEQLARAGFYTDWALGEGDK
VKCFHCGGGLRPSEDPWEQHARWYPGCRYLLEQRGQEYINNIHLTHS.

Peptide inhibitors of caspase-9 include those amino acid sequences that retain certain structural and functional features of the above-identified XBIR3 peptides, yet differ from the identified inhibitors' amino acid sequences at one or more positions. Such variants can be prepared by substituting, deleting, or adding amino acid residues from the original sequences via methods known in the art.

In certain embodiments, such substantially similar sequences include sequences that incorporate conservative amino acid substitutions. As used herein, a “conservative amino acid substitution” is intended to include a substitution in which the amino acid residue is replaced with an amino acid residue having a similar side chain. Families of amino acid residues having similar side chains have been defined in the art, including: basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine); nonpolar side chains (e.g., alanine, valine, leucine, isoleucine, proline, phenylalanine, methionine, tryptophan); β-branched side chains (e.g., threonine, valine, isoleucine); and aromatic side chains (e.g., tyrosine, phenylalanine, tryptophan, histidine). Other generally preferred substitutions involve replacement of an amino acid residue with another residue having a small side chain, such as alanine or glycine. Amino acid substituted peptides can be prepared by standard techniques, such as automated chemical synthesis.

In certain embodiments, a peptide inhibitor of caspase-9 of the present disclosure is at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% homologous to the amino acid sequences of XBIR3 identified above. As used herein, the percent homology between two amino acid sequences may be determined using standard software such as BLAST or FASTA. The effect of the amino acid substitutions on the ability of the synthesized peptide inhibitor of caspase-9 to inhibit caspase-9 can be tested using the methods disclosed in Examples section, below.

### Inhibitor-Cell Penetrating Peptide Conjugates

In certain embodiments of the present disclosure, the caspase-9 signaling pathway inhibitor is conjugated to a cell penetrating peptide, typically via a disulfide bond, to form an inhibitor-cell penetrating peptide conjugate.

As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell. In certain embodiments, the cell-penetrating peptide used in the membrane-permeable complex of the present disclosure preferably comprises at least one non-functional cysteine residue, which is either free or derivatized to form a disulfide link with the caspase-9 signaling pathway inhibitor, which has been modified for such linkage. Representative amino acid motifs conferring such properties are listed in U.S. Pat. No. 6,348,185, the contents of which are expressly incorporated herein by reference. The cell-penetrating peptides of the present disclosure may include, but are not limited to, Penetratin1, transportan, pIsl, TAT(48-60), pVEC, MTS, and MAP.

The cell-penetrating peptides of the present disclosure include those sequences that retain certain structural and functional features of the identified cell-penetrating peptides, yet differ from the identified peptides' amino acid sequences at one or more positions. Such polypeptide variants can be prepared by substituting, deleting, or adding amino acid residues from the original sequences via methods known in the art.

In certain embodiments, such substantially similar sequences include sequences that incorporate conservative amino acid substitutions, as described above in connection with caspase-9 inhibitors. In certain embodiments, a cell-penetrating peptide of the present disclosure is at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% homologous to the amino acid sequence of the identified peptide and is capable of mediating cell penetration. The effect of the amino acid substitutions on the ability of the synthesized peptide to mediate cell penetration can be tested using the methods disclosed in Examples section, below.

In certain embodiments of the present disclosure, the cell-penetrating peptide of the membrane-permeable complex is Penetratin1, comprising the peptide sequence C(NPys)-RQIKIWFQNRRMKWKK (SEQ ID NO: 9), or a conservative variant thereof. As used herein, a “conservative variant” is a peptide having one or more amino acid substitutions, wherein the substitutions do not adversely affect the shape—or, therefore, the biological activity (i.e., transport activity) or membrane toxicity—of the cell-penetrating peptide.

Other non-limiting embodiments of the present disclosure involve the use of the following exemplary cell permeant molecules: RL16 (H-RRLRRLLRRLLRRLRR-OH) (SEQ ID NO: 10), a sequence derived from Penetratin1 with slightly different physical properties; and RVG-RRRRRRRRR (SEQ ID NO: 11), a rabies virus sequence which targets neurons.

In certain alternative non-limiting embodiments of the present disclosure, the cell-penetrating peptide of the membrane-permeable complex is a cell-penetrating peptide selected from the group consisting of: transportan, pISl, Tat(48-60), pVEC, MAP, and MTS. Transportan is a 27-amino-acid long peptide containing 12 functional amino acids from the amino terminus of the neuropeptide galanin, and the 14-residue sequence of mastoparan in the carboxyl terminus, connected by a lysine. It includes the amino acid sequence GWTLNSAGYLLGKINLKALAALAKKIL (SEQ ID NO: 12), or a conservative variant thereof.

pIsl is derived from the third helix of the homeodomain of the rat insulin 1 gene enhancer protein. pIsl includes the amino acid sequence PVIRVW FQNKRCKDKK (SEQ ID NO: 13), or a conservative variant thereof.

Tat is a transcription activating factor, of 86-102 amino acids, that allows translocation across the plasma membrane of an HIV-infected cell, to transactivate the viral genome. A small Tat fragment, extending from residues 48-60, has been determined to be responsible for nuclear import; it includes the amino acid sequence GRKKRRQRRRPPQ (SEQ ID NO: 14), or a conservative variant thereof.

pVEC is an 18-amino-acid-long peptide derived from the murine sequence of the cell-adhesion molecule, vascular endothelial cadherin, extending from amino acid 615-632. pVEC includes the amino acid sequence LLIILRRRIRKQAHAH (SEQ ID NO: 15), or a conservative variant thereof.

MTSs, or membrane translocating sequences, are those portions of certain peptides which are recognized by the acceptor proteins that are responsible for directing nascent translation products into the appropriate cellular organelles for further processing. An MTS of particular relevance is MPS peptide, a chimera of the hydrophobic terminal domain of the viral gp41 protein and the nuclear localization signal from simian virus 40 large antigen; it represents one combination of a nuclear localization signal and a membrane translocation sequence that is internalized independent of temperature, and functions as a carrier for oligonucleotides. MPS includes the amino acid sequence GALFLGWLGAAGSTMGAWSQPKKKRKV (SEQ ID NO: 16), or a conservative variant thereof.

Model amphipathic peptides, or MAPs, form a group of peptides that have, as their essential features, helical amphipathicity and a length of at least four complete helical turns. An exemplary MAP comprises the amino acid sequence KLALKLALKALKAALKLA (SEQ ID NO: 17)-amide, or a conservative variant thereof.

In certain embodiments, the cell-penetrating peptides and the caspase-9 signaling pathway inhibitors described above are covalently bound to form conjugates. In certain embodiments the cell-penetrating peptide is operably linked to a caspase-9 inhibitor via recombinant DNA technology. For example, in embodiments where the caspase-9 signaling pathway inhibitor is a caspase-9 inhibitor, a nucleic acid sequence encoding that caspase-9 inhibitor can be introduced either upstream (for linkage to the amino terminus of the cell-penetrating peptide) or downstream (for linkage to the carboxy terminus of the cell-penetrating peptide), or both, of a nucleic acid sequence encoding the caspase-9 inhibitor of interest. Such fusion sequences including both the caspase-9 inhibitor encoding nucleic acid sequence and the cell-penetrating peptide encoding nucleic acid sequence can be expressed using techniques well known in the art.

In certain embodiments the caspase-9 signaling pathway inhibitor can be operably linked to the cell-penetrating peptide via a non-covalent linkage. In certain embodiments such non-covalent linkage is mediated by ionic interactions, hydrophobic interactions, hydrogen bonds, or van der Waals forces.

In certain embodiments the caspase-9 signaling pathway inhibitor is operably linked to the cell penetrating peptide via a chemical linker. Examples of such linkages typically incorporate 1-30 nonhydrogen atoms selected from the group consisting of C, N, O, S and P. Exemplary linkers include, but are not limited to, a substituted alkyl or a substituted cycloalkyl. Alternately, the heterologous moiety may be directly attached (where the linker is a single bond) to the amino or carboxy terminus of the cell-penetrating peptide. When the linker is not a single covalent bond, the linker may be any combination of stable chemical bonds, optionally including, single, double, triple or aromatic carbon-carbon bonds, as well as carbon-nitrogen bonds, nitrogen-nitrogen bonds, carbon-oxygen bonds, sulfur-sulfur bonds, carbon-sulfur bonds, phosphorus-oxygen bonds, phosphorus-nitrogen bonds, and nitrogen-platinum bonds. In certain embodiments, the linker incorporates less than 20 nonhydrogen atoms and are composed of any combination of ether, thioether, urea, thiourea, amine, ester, carboxamide, sulfonamide, hydrazide bonds and aromatic or heteroaromatic bonds. In certain embodiments, the linker is a combination of single carbon-carbon bonds and carboxamide, sulfonamide or thioether bonds.

In certain embodiments the caspase-9 signaling inhibitor is encapsulated into a nano-carrier, such as magnetic iron oxide nanoparticles, and the nano-carrier is operably linked to the nano-carrier. In certain embodiments, pH-sensitive nano-carriers can be used, which may employ the use of acid-sensitive linkages such as benzoic imine or hydrazine bonds. In certain embodiments, the nano-carrier can be a nano-carrier a liposome, a lipid based nano-particle, a synthetic polymer, a dendrimer, a silica-based nano-particle, or carbon nano-materials. Use of nano-carriers has been well studied and Yu et al., Int J Mol Sci. 2016 Nov; 17(11): 1892 further discusses their uses and drug delivery capability.

A general strategy for conjugation involves preparing the cell-penetrating peptide and the caspase-9 signaling pathway inhibitor components separately, wherein each is modified or derivatized with appropriate reactive groups to allow for linkage between the two. The modified caspase-9 signaling pathway inhibitor is then incubated together with a cell-penetrating peptide that is prepared for linkage, for a sufficient time (and under such appropriate conditions of temperature, pH, molar ratio, etc.) as to generate a covalent bond between the cell-penetrating peptide and the caspase-9 signaling pathway inhibitor molecule.

Numerous methods and strategies of conjugation will be readily apparent to one of ordinary skill in the art, as will the conditions required for efficient conjugation. By way of example only, one such strategy for conjugation is described below, although other techniques, such as the production of fusion proteins or the use of chemical linkers is within the scope of the present disclosure.

In certain embodiments, when generating a disulfide bond between the caspase-9 signaling pathway inhibitor molecule and the cell-penetrating peptide of the present disclosure, the caspase-9 signaling pathway inhibitor molecule can be modified to contain a thiol group, and a nitropyridyl leaving group can be manufactured on a cysteine residue of the cell-penetrating peptide. Any suitable bond (e.g., thioester bonds, thioether bonds, carbamate bonds, etc.) can be created according to methods generally and well known in the art. Both the derivatized or modified cell-penetrating peptide, and the modified caspase-9 signaling pathway inhibitor are reconstituted in RNase/DNase sterile water, and then added to each other in amounts appropriate for conjugation (e.g., equimolar amounts). The conjugation mixture is then incubated for 60 min at 37° C., and then stored at 4° C. Linkage can be checked by running the vector-linked caspase-9 signaling pathway inhibitor molecule, and an aliquot that has been reduced with DTT, on a 15% non-denaturing PAGE. Caspase-9 signaling pathway inhibitor molecules can then be visualized with the appropriate stain.

In certain embodiments, the present disclosure is directed to a Penetratin1 1-XBIR3 (Pen1-XBIR3) conjugate. In certain of such embodiments, the sequence of the Pen-1-XBIR3 is: C(NPys)-RQIKIWFQNRRMKWKK-s-s-MGSSHHHHHHSSGLVPRGSHMSTNTCLPRNPSMADYEARIFTFGTWIYSVNKE QLARAGFYTDWALGEGDKVKCFHCGGGLRPSEDPWEQHARWYPGCRYLLEQ RGQEYINNIHLTHS (SEQ ID NO: 18). In other of such embodiments, the sequence of the Pen-1-XBIR3 is: C(NPys)-RQIKIWFQNRRMKWKK-s-s-MGSSSSGLVPRGSHMSTNTCLPRNPSMADYEARIFTFGTWIYSVNKEQLARAG FYTDWALGEGDKVKCFHCGGGLRPSEDPWEQHARWYPGCRYLLEQRGQEYIN NIHLTHS (SEQ ID NO: 19). In other of such embodiments, the sequence of the Pen-1-XBIR3 is: C(NPys)-RQIKIWFQNRRMKWKK-s-s-SSGLVPRGSHMSTNTCLPRNPSMADYEARIFTFGTWIYSVNKEQLARAGFYTD WALGEGDKVKCFHCGGGLRPSEDPWEQHARWYPGCRYLLEQRGQEYINNIHL THS (SEQ ID NO: 20). In other of such embodiments, the sequence of the Pen-1-XBIR3 is: C(NPys)-RQIKIWFQNRRMKWKK-s-s-MSTNTCLPRNPSMADYEARIFTFGTWIYSVNKEQLARAGFYTDWALGEGDKV KCFHCGGGLRPSEDPWEQHARWYPGCRYLLEQRGQEYINNIHLTHS (SEQ ID NO: 21). In other of such embodiments, the sequence of the Pen1-XBIR3 is: C(NPys)-RQIKIWFQNRRMKWKK-s-s-MGSSHHHHHHSSGLVPRGSHMSTNTLPRNPSMADYEARIFTFGTWIYSVNKEQ LARAGFYTDWALGEGDKVKCFHCGGGLRPSEDPWEQHARWYPGCRYLLEQR GQEYINNIHLTHS (SEQ ID NO: 22)

In other of such embodiments, the sequence of the Pen1-XBIR3 is: C(NPys)-RQIKIWFQNRRMKWKK-s-s-MGSSSSGLVPRGSHMSTNTLPRNPSMADYEARIFTFGTWIYSVNKEQLARAGF YTDWALGEGDKVKCFHCGGGLRPSEDPWEQHARWYPGCRYLLEQRGQEYIN NIHLTHS (SEQ ID NO: 23). In other of such embodiments, the sequence of the Pen1-XBIR3 is: C(NPys)-RQIKIWFQNRRMKWKK-s-s-SSGLVPRGSHMSTNTLPRNPSMADYEARIFTFGTWIYSVNKEQLARAGFYTDW ALGEGDKVKCFHCGGGLRPSEDPWEQHARWYPGCRYLLEQRGQEYINNIHLT HS (SEQ ID NO: 24). In other of such embodiments, the sequence of the Pen1-XBIR3 is: C(NPys)-RQIKIWFQNRRMKWKK-s-s-MSTNTLPRNPSMADYEARIFTFGTWIYSVNKEQLARAGFYTDWALGEGDKVK CFHCGGGLRPSEDPWEQHARWYPGCRYLLEQRGQEYINNIHLTHS (SEQ ID NO: 25).

### Methods of Administration

In certain embodiments, the caspase-9 signaling pathway inhibitors or conjugates of the present disclosure are formulated for retinal administration, for example as eye drops or a topical ocular formulation. For administration via eye drops, a solution or suspension containing the caspase-9 signaling pathway inhibitor or conjugate can be formulated for direct application to the retina by conventional means, for example with a dropper, pipette or spray. In certain embodiments, the caspase-9 signaling pathway inhibitor or conjugate of the present disclosure is formulated in isotonic saline. In certain embodiments, the caspase-9 signaling pathway inhibitor or conjugate of the present disclosure is formulated in isotonic saline at or about pH 7.4.

In certain embodiments, the caspase-9 signaling pathway inhibitors or conjugates of the present disclosure are formulated for intranasal delivery, for example as a nasal spray. In certain other embodiments, the caspase-9 signaling pathway inhibitors or conjugates of the present disclosure are formulated for systemic delivery, for example as a intravenous injection or oral medication.

To facilitate delivery to a cell, tissue, or subject, the caspase-9 signaling pathway inhibitor, or conjugate thereof, of the present disclosure may, in various compositions, be formulated with a pharmaceutically-acceptable carrier, excipient, or diluent. The term “pharmaceutically-acceptable”, as used herein, means that the carrier, excipient, or diluent of choice does not adversely affect either the biological activity of the caspase-9 signaling pathway inhibitor or conjugate or the biological activity of the recipient of the composition. Suitable pharmaceutical carriers, excipients, and/or diluents for use in the present disclosure include, but are not limited to, lactose, sucrose, starch powder, talc powder, cellulose esters of alkonoic acids, magnesium stearate, magnesium oxide, crystalline cellulose, methyl cellulose, carboxymethyl cellulose, gelatin, glycerin, sodium alginate, gum arabic, acacia gum, sodium and calcium salts of phosphoric and sulfuric acids, polyvinylpyrrolidone and/or polyvinyl alcohol, saline, and water.

In accordance with the methods of the present disclosure, the quantity of the caspase-9 signaling pathway inhibitor or conjugate thereof that is administered to a cell, tissue, or subject should be an effective amount.

### EXAMPLES

### Example 1: Phenotypic Characterization of Mitochondrial Complex I Deficiency Retinal Pathology

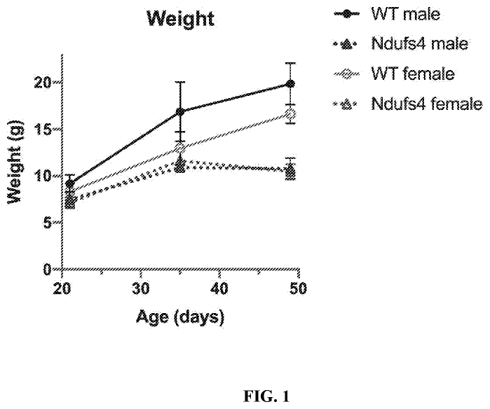

Two sets of mice were used for phenotypic characterization. The first set were NDUFS4 KO mice, in which the NDUFS4 gene is not operable, and thus the NDUFS4 enzyme is not produced. Such mice are a model for mitochondrial complex I deficiency in humans. The second set of mice were simply wild type. The mice were assessed at the early stage (3 weeks postnatal), mid-stage (5 weeks postnatal), and late stage (7 weeks postnatal) of the disease. The weights of the mice were taken at the various stages and are presented in FIG. 1. The mice were sacrificed at day 50 for immunohistochemical analysis.

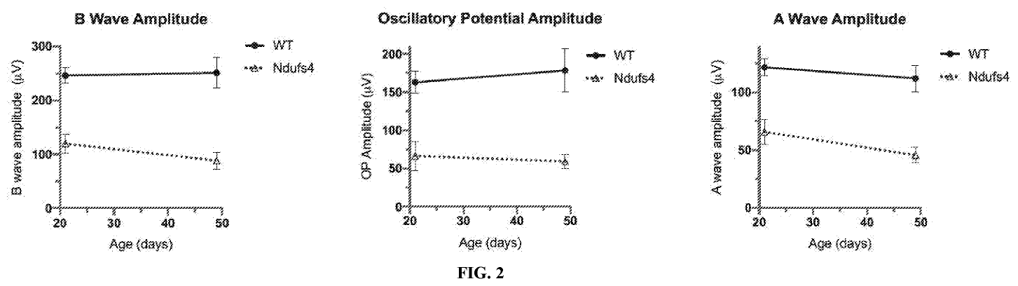

An electroretinogram of the mice was taken at the early stage and late stage. The readouts from the electroretinogram are presented in FIG. 2. The A wave measures photoreceptor activity, and the B wave and oscillatory potentials measure activity of the inner retinal neurons. A deficit in ERG amplitude can be caused by neurodegeneration or energy deficits.

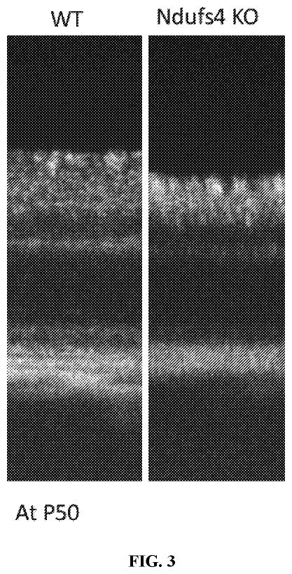

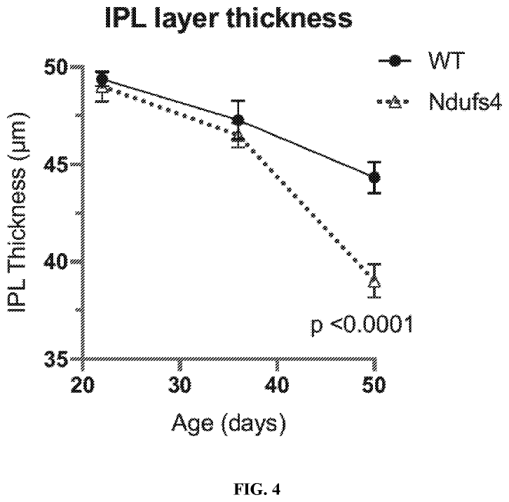

Optical coherence tomography images of the mice were taken on weeks 3, 5, and 7. Two of the optical coherence tomography images from week 7 are presented on FIG. 3. FIG. 4 presents a graph of the inner plexiform layer thickness over time for the wild type and NDUFS4 KO groups.

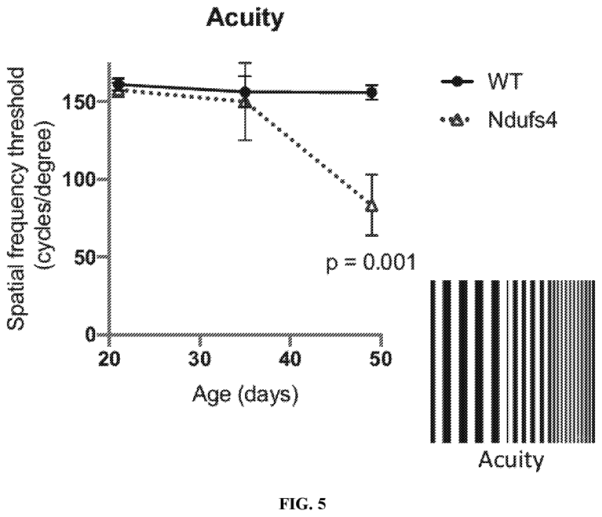

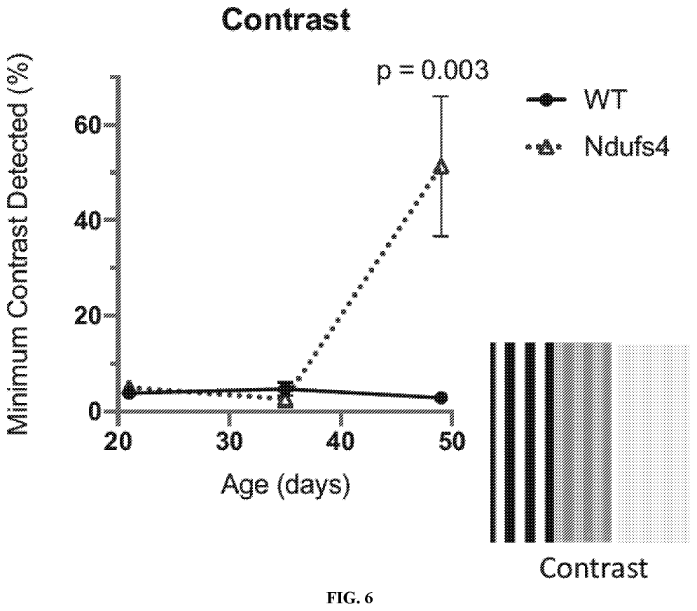

Optomotor tracking of visual acuity and contrast sensitivity was also performed on the mice. FIG. 5 presents the visual acuity sensitivity data, which displays the spatial frequency threshold of both mice groups at the early stage, mid-stage, and late stage. FIG. 6 presents the visual contrast sensitivity data, which displays the minimum contrast detected of both mice groups at the early stage, mid-stage, and late stage.

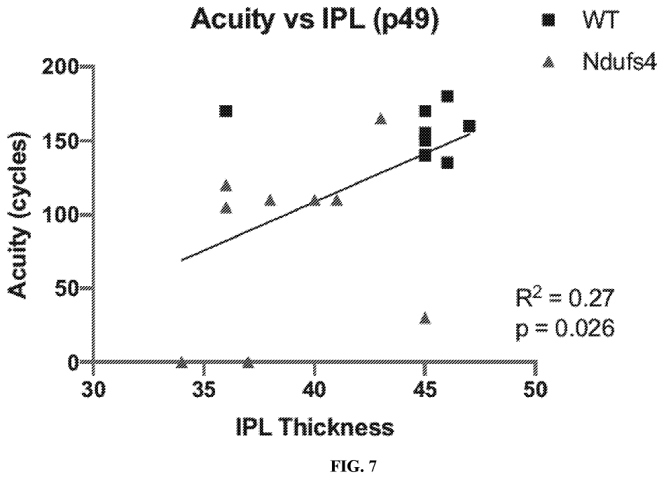

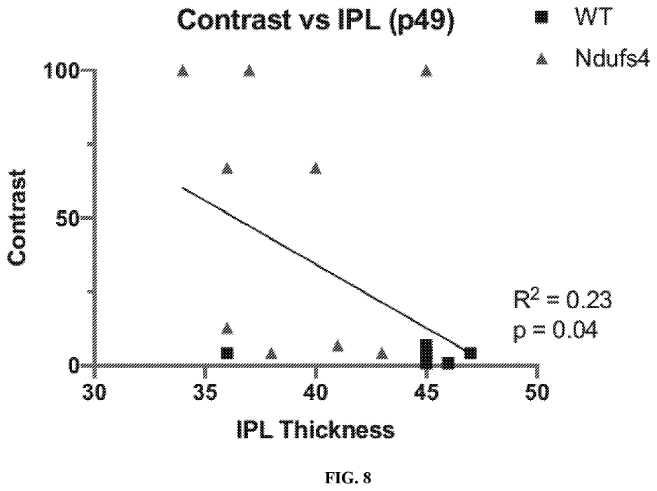

A correlation was found between vision deficits and inner plexiform layer thickness, which was present for deficits in acuity and contrast. FIG. 7 presents acuity data versus inner plexiform layer thickness and FIG. 8 presents contrast data versus inner plexiform layer thickness.

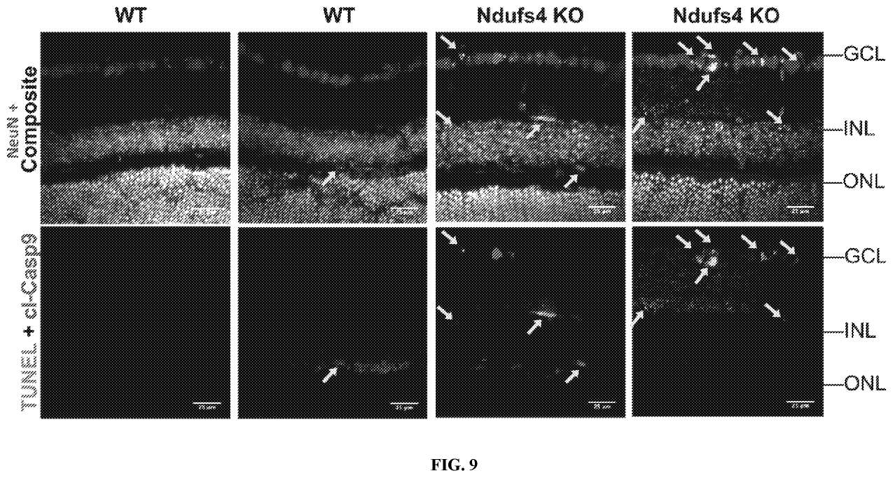

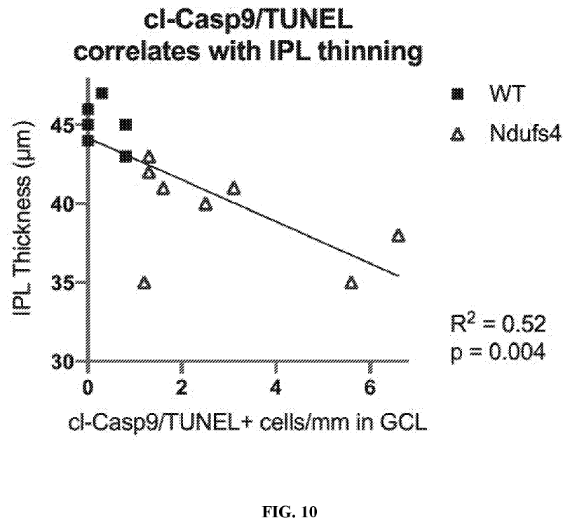

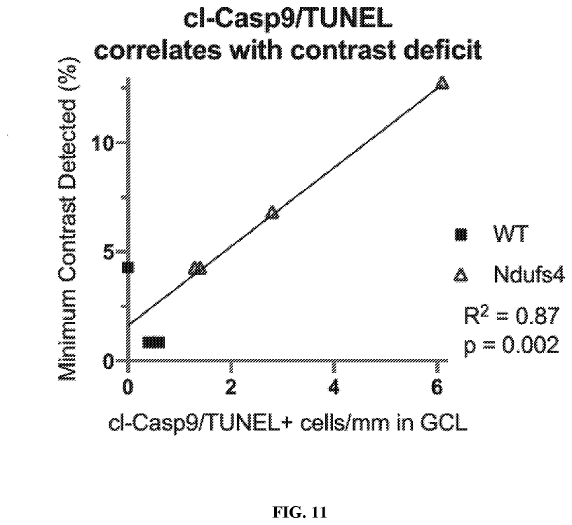

At week 7, TUNEL staining was performed looking for the presence of cl-Casp-9 in the retina of the two sets of mice. FIG. 9. Represents the TUNEL presents images of the TUNEL staining. Data collected looking for the presence of cl-Casp-9 from the TUNEL staining and was plotted against the IPL thickness and contract sensitivity data. Higher levels of cl-Casp-9 were found to correlate with a decrease in IPL thickness as shown in FIG. 10. Further, higher levels of cl-Casp-9 were found to correlate with a decrease in contrast sensitivity, which is shown in FIG. 11. Thus, the presence of higher levels of cl-Casp-9 were found to be directly correlated with the symptoms and presence of a mitochondrial complex I deficiency.

### Example 2: Use of Caspase-9 Signaling Pathway Inhibitors to Treat Mitochondrial Complex I Deficiency

A second study was conducted using NDUFS4 KO mice and wild type C57B1/6J mice. The NDUFS4 KO mice were obtained by breeding NDUFS4 heterozygote mice purchased from Jackson labs (Stock No: 027058). The wild type mice were also purchased from Jackson labs (Stock No: 00664). Both male and female mice were used in the study.

To prepare the Pen1-XBir3 and Pen1-mutXBir3, His-tagged XBir3 and mutXBir3 (inactive mutant) were expressed in Escherichia coli and purified by nickel column. Pen1 (PolyPeptide Group) was mixed at a 1:1 molar ratio with purified XBir3 or mutXBir3 (160 μM concentration) and incubated for 30 minutes to 1 hour at room temperature to generate disulfide-linked Pen1-XBir3 and Pen1-mutXBir3.

For treating the mice, NDUFS4 KO littermates were randomly assigned to treatment with either 10 μg Pen1-XBir3 or 10 μg Pen1-mutXBir3 topical daily eye-drops in both eyes from postnatal day 35 through postnatal day 49.

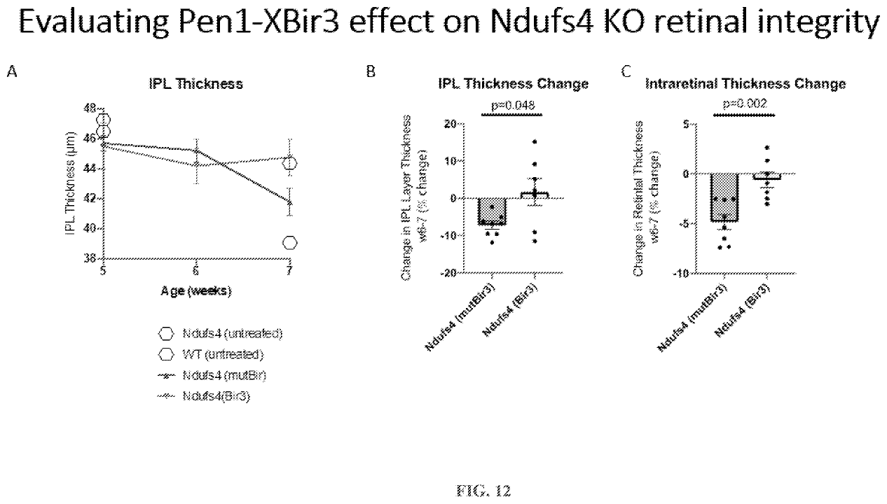

OCT images were captured using the Phoenix Micron IV image-guided OCT system at postnatal day 35, 42, and 49. For each eye, two vertical and two horizontal OCT scans were captured approximately 2 optic disc lengths from the optic nerve. For each eye, four OCT images were averaged to generate mean retinal thickness values. Segmentation of individual retinal layers was generated using InSight software, and average layer thicknesses were calculated in Excel. Intraretinal thickness was measured from GCL to the outer segment (OS). The OCT imaging captured IPL and intraretinal thickness in the mice, the change in IPL thickness throughout the study is seen in FIG. 12. FIG. 12. shows a decrease in retinal thinning, this decrease in retinal thinning is by at least 5%.

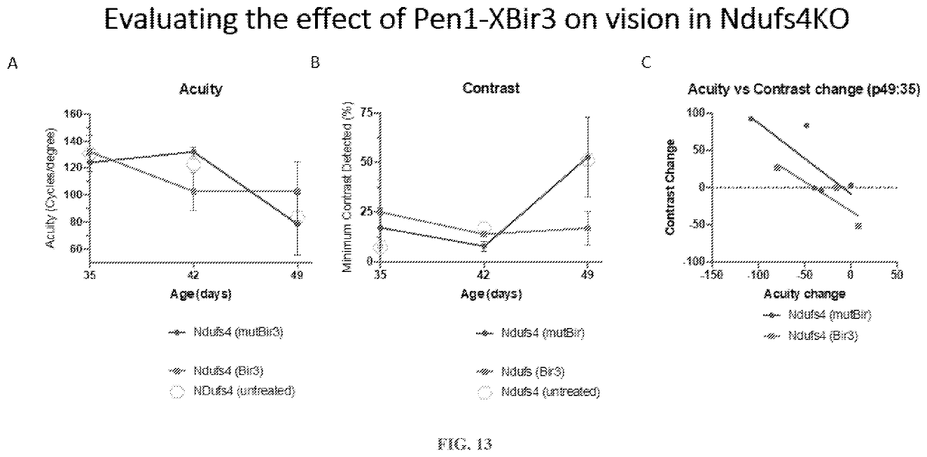

Vision acuity to determine contrast and acuity sensitivity was assessed in awake unrestrained animals by OMRs in the OptoDrum (Striatech) automated optomotor system at postnatal day 35, 42, and 49. The acuity and contrast captured is seen in FIG. 13.

All treatment, imaging, and data analysis was performed by investigators blinded to the treatment groups.

The data from Example 1 shows that higher levels of cl-Casp-9 are present in mice having mitochondrial complex I deficiency. Further, higher concentrations of cl-Casp-9 has been found to be directly correlated with symptom severity in mice having mitochondrial complex I deficiency. Based on this data, it was contemplated that Pen1-XBir3 will treat mitochondrial complex I deficiency, as Pen1-XBir3 is an inhibitor of caspase-9. Further, it is contemplated that any caspase-9 signaling pathway inhibitor will treat mitochondrial Complex I deficiency.

Example 2 confirms that Pen1-XBir3 will treat mitochondrial complex I deficiency, as well as prevent retinal degradation in NDUFS4 KO mice. The IPL thinning in patients with complex I deficiency reflects loss of ganglion cells. Prevention of IPL thinning in the NDUFS4 KO model suggests that caspase-9 inhibition prevents ganglion cell death caused by complex I deficiency. Further, prevention of retinal atrophy in eyes treated with Pen1-XBir3 indicates that caspase-9 inhibition reduces retinal cell death caused by complex I deficiency. Caspase-9 inhibition with Pen1-XBir3 prevents onset of contrast sensitivity deficits in NDUFS4 KO mouse model of complex I deficiency, supporting functional vision preservation.

The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims.

Patents, patent applications, publications, procedures, and the like are cited throughout this application, the disclosures of which are incorporated herein by reference in their entireties.

1. A method for treating a mitochondrial complex I deficiency, the method comprising administering to an eye containing an inner plexiform layer and a ganglion cell layer of a patient having a mitochondrial complex I deficiency an amount of a caspase-9 signaling pathway inhibitor comprising XBIR3 conjugated to a cell-penetrating peptide effective to treat the mitochondrial complex I deficiency by decreasing the amount of 4-Hydroxynonenal (4-HNE) in the inner plexiform layer of the eye of the patient by more than 5%, reducing retinal thinning by at least 5%, decreasing the amount of cl-Casp-9 in the inner plexiform layer of the eye of the patient by more than 5%, or decreasing the rate of ganglion cell death in the ganglion cell layer as measured by an improvement of 5% minimum contrast detected as measured by contrast sensitivity testing.

2. (canceled)

3. The method of claim 1, wherein administering to the eye of the patient comprises administering an eye drop comprising the caspase-9 signaling pathway inhibitor comprising XBIR3 conjugated to the cell-penetrating peptide.

4. The method of claim 1, wherein administering to the eye of the patient comprises administering a topical ophthalmic ointment comprising the caspase-9 signaling pathway inhibitor comprising XBIR3 conjugated to the cell-penetrating peptide.

5. The method of claim 1, wherein administering comprises administering systemically to the patient.

6. The method of claim 1, wherein administering comprises administering via intranasal delivery to the patient.

7. The method of claim 1, wherein the cell-penetrating peptide is selected from the group consisting of Penetratin1, transportan, pISl, Tat (48-60) , pVEC, MAP, and MTS.

8. The method of claim 1, wherein the XBIR3 is conjugated to the cell-penetrating peptide via a disulfide bond.

9-11. (canceled)

12. The method of claim 1, wherein the amount of the caspase-9 signaling pathway inhibitor comprising XBIR3 conjugated to the cell-penetrating peptide effective to treat the mitochondrial complex I deficiency increases the patient's acuity sensitivity by more than 10 cycle/degrees as measured by visual acuity testing.

13. The method of claim 1, wherein the amount of the caspase-9 signaling pathway inhibitor comprising XBIR3 conjugated to the cell-penetrating peptide effective to treat the mitochondrial complex I deficiency increases the patient's contrast sensitivity as measured by an improvement of 10% minimum contrast detected as measured by contrast sensitivity testing.

14. The method of claim 1, wherein the caspase-9 signaling pathway inhibitor comprising XBIR3 conjugated to the cell-penetrating peptide is administered at a concentration between 0.1 μM and 1,000 μM, inclusive.

15. The method of claim 1, wherein XBIR3 is indirectly conjugated to the cell-penetrating peptide, by encapsulating XBIR3 within a nano-carrier, and the nano-carrier is conjugated to the cell-penetrating a cell penetrating peptide.

16. (canceled)

17. The method of claim 1, wherein the XBIR3 has a sequence selected from the group consisting of SEQ ID NOs: 1-8.

18. The method of claim 1, wherein the cell-penetrating peptide has a sequence selected from the group consisting of SEQ ID NOs: 9-17.

19. The method of claim 1, wherein the caspase-9 signaling pathway inhibitor comprising XBIR3 conjugated to the cell-penetrating peptide has a sequence selected from:
C(NPys)-RQIKIWFQNRRMKWKK-s-s-
(SEQ ID NO: 18)
MGSSHHHHHHSSGLVPRGSHMSTNTCLPRNPSMADYEARIFTFGTWIYSV
NKEQLARAGFYTDWALGEGDKVKCFHCGGGLRPSEDPWEQHARWYPGCRY
LLEQRGQEYINNIHLTHS;
C(NPys)-RQIKIWFQNRRMKWKK-s-s-
(SEQ ID NO: 19)
MGSSSSGLVPRGSHMSTNTCLPRNPSMADYEARIFTFGTWIYSVNKEQLA
RAGFYTDWALGEGDKVKCFHCGGGLRPSEDPWEQHARWYPGCRYLLEQRG
QEYINNIHLTHS;
C(NPys)-RQIKIWFQNRRMKWKK-s-s-
(SEQ ID NO: 20)
SSGLVPRGSHMSTNTCLPRNPSMADYEARIFTFGTWIYSVNKEQLARAGF
YTDWALGEGDKVKCFHCGGGLRPSEDPWEQHARWYPGCRYLLEQRGQEYI
NNIHLTHS;
(NPys)-RQIKIWFQNRRMKWKK-s-s-
(SEQ ID NO: 21)
MSTNTCLPRNPSMADYEARIFTFGTWIYSVNKEQLARAGFYTDWALGEGD
KVKCFHCGGGLRPSEDPWEQHARWYPGCRYLLEQRGQEYINNIHLTHS;
C(NPys)-RQIKIWFQNRRMKWKK-s-s-
(SEQ ID NO: 22)
MGSSHHHHHHSSGLVPRGSHMSTNTLPRNPSMADYEARIFTFGTWIYSVN
KEQLARAGFYTDWALGEGDKVKCFHCGGGLRPSEDPWEQHARWYPGCRYL
LEQRGQEYINNIHLTHS;
C(NPys)-RQIKIWFQNRRMKWKK-s-s-
(SEQ ID NO: 23)
MGSSSSGLVPRGSHMSTNTLPRNPSMADYEARIFTFGTWIYSVNKEQLAR
AGFYTDWALGEGDKVKCFHCGGGLRPSEDPWEQHARWYPGCRYLLEQRGQ
EYINNIHLTHS;
C(NPys)-RQIKIWFQNRRMKWKK-s-s-
(SEQ ID NO: 24)
SSGLVPRGSHMSTNTLPRNPSMADYEARIFTFGTWIYSVNKEQLARAGFY
TDWALGEGDKVKCFHCGGGLRPSEDPWEQHARWYPGCRYLLEQRGQEYIN
NIHLTHS;
and
C(NPys)-RQIKIWFQNRRMKWKK-s-s-
(SEQ ID NO: 25)
MSTNTLPRNPSMADYEARIFTFGTWIYSVNKEQLARAGFYTDWALGEGDK
VKCFHCGGGLRPSEDPWEQHARWYPGCRYLLEQRGQEYINNIHLTHS.

20. The method of claim 1, wherein the cell-penetrating peptide is Penetratin1.

21. The method of claim 1, wherein administering comprises a single dose.

22. The method of claim 1, wherein administering comprises multiple doses.

23. The method of claim 22, wherein the multiple doses are administered at intervals of 6 times per 24 hours, 4 times per 24 hours, 3 times per 24 hours, 2 times per 24 hours, 1 time per 24 hours, 1 time every other day, 1 time every 3 days, 1 time every 4 days, 1 time per week, 2 times per week, or 3 times per week.

24. The method of claim 1, wherein the amount of the caspase-9 signaling pathway inhibitor comprising XBIR3 conjugated to the cell-penetrating peptide effective to treat the mitochondrial complex I deficiency decreases apoptosis in the inner plexiform layer of the patient.

25. The method of claim 1, wherein the amount of the caspase-9 signaling pathway inhibitor comprising XBIR3 conjugated to the cell-penetrating peptide effective to treat the mitochondrial complex I deficiency increases the thickness of the inner plexiform layer of the eye.
